(19) United States
(12) Patent Application Publication
(10) Pub. No.: US 2026/0284382 A1
(43) Pub. Date: Sep. 24, 2026

(54) FLEXIBLE NEURAL ELECTRODE ASSEMBLY

(71) Applicant: BCIFlex Medical Technology Co., Ltd., Beijing (CN)

(72) Inventors: Dawei WANG, Beijing (CN); Guiqiang YANG, Beijing (CN); Huihui TIAN, Beijing (CN); Ye TIAN, Beijing (CN); Qian LI, Beijing (CN)

(21) Appl. No.: 19/573,311

(22) Filed: Mar. 20, 2026

(30) Foreign Application Priority Data
Mar. 21, 2025 (CN) 202510336199.3

(51) Int. Cl. A61N 1/05

(52) U.S. Cl. CPC A61N 1/0539

(57) ABSTRACT

The present application provides a flexible neural electrode assembly which includes a flexible electrode, a traction member, an auxiliary fixing assembly, and an elastic sealing member. The flexible electrode includes a proximal contact portion, a lead connection portion, a distal electrode portion, and an auxiliary implantation portion. The flexible electrode and traction member both penetrate through the auxiliary fixing assembly for implantation into the target tissue. The elastic sealing member is sleeved outside the flexible electrode and the traction member and configured to abut against the auxiliary fixing assembly at least after the distal electrode portion is implanted into the target tissue to a preset depth, and to deform under the action of the auxiliary fixing assembly to compress the flexible electrode, such that the distal electrode portion of the flexible electrode is fixed at the preset depth, and the flexible electrode is sealingly connected to the auxiliary fixing assembly via the elastic sealing member.

### TECHNICAL FIELD

The present application relates to the technical field of biomedical engineering, specifically, to a flexible neural electrode assembly.

### BACKGROUND

Flexible neural electrodes have important application value in neuroscience research and neural electrical stimulation therapy. In the prior art, in order to enable precise implantation of flexible neural electrodes, a traction device or an auxiliary fixing portion is usually employed in conjunction. However, these technical solutions still have some shortcomings in practical applications. For example, the consideration for the sealing design for flexible neural electrodes in the prior art is insufficient, which is prone to lead to the risk of leakage or contamination of body fluids (such as blood, cerebrospinal fluid, etc.) or gases, further increasing the probability of postoperative complications for patients. In addition, the problem of unstable fixation of flexible neural electrodes after implantation has not been fully addressed. Especially, in dynamic environments, the flexible neural electrodes may shift, which affects the therapeutic effect. Particularly, the lack of effective restrictions on the implantation depth of the flexible neural electrodes during the implantation is likely to cause the flexible neural electrode to be implanted too deeply or away from the target area.

### SUMMARY

In response to the aforementioned technical problems in the prior art, the present application provides a flexible neural electrode assembly, which enables effective limitation of the implantation depth of the flexible electrode and can achieve effective sealing of the wound where the flexible electrode is implanted through the synergistic action of an elastic sealing member and an auxiliary fixing assembly.

The present application provides a flexible neural electrode assembly. The flexible neural electrode assembly comprises a flexible electrode, a traction member, an auxiliary fixing assembly, and an elastic sealing member. The flexible electrode is made of a flexible material and comprises a proximal contact portion, a lead connection portion, a distal electrode portion, and an auxiliary implantation portion sequentially connected along a length direction of the flexible electrode. The distal electrode portion and the auxiliary implantation portion are used for being implanted into a target tissue, and the distal electrode portion comprises at least one electrode site for applying electrical stimulation to the target tissue and/or acquiring potential signals of the target tissue. The traction member acts on the auxiliary implantation portion to implant the flexible electrode toward a target area via the auxiliary implantation portion. The auxiliary fixing assembly is connected to a surface of the target tissue and/or an accessory anatomical structure of the target tissue, and the flexible electrode and traction member both penetrate through the auxiliary fixing assembly for implantation into the target tissue. The elastic sealing member is sleeved outside the flexible electrode and the traction member. The elastic sealing member is configured to abut against the auxiliary fixing assembly at least after the distal electrode portion is implanted into the target tissue to a preset depth, and to deform under the action of the auxiliary fixing assembly to compress the flexible electrode, such that the distal electrode portion of the flexible electrode is fixed at the preset depth, and the flexible electrode is further sealingly connected to the auxiliary fixing assembly via the elastic sealing member.

In some embodiments, the auxiliary fixing assembly comprises a first fixing member connected to the surface of the target tissue and/or the accessory anatomical structure of the target tissue, and the first fixing member is provided with a first through channel. The flexible electrode and traction member are implanted into the target tissue through the first through channel, and the elastic sealing member abuts against a proximal end of the first fixing member.

In some embodiments, the elastic sealing member has a second through channel through which the flexible electrode penetrates, and the second through channel is configured to deform toward the flexible electrode under the action of the auxiliary fixing assembly to abut against the flexible electrode.

In some embodiments, the elastic sealing member further has a third through channel through which the traction member penetrates, and the second through channel and the third through channel are in communication with each other or separately arranged.

In some embodiments, the elastic sealing member is configured to move relative to the flexible electrode along the length direction of the flexible electrode when subjected to a force exceeding a first threshold.

In some embodiments, the second through channel and/or the third through channel is slit-shaped.

In some embodiments, the second through channel of the elastic sealing member is configured to apply a first friction force to the flexible electrode penetrating therethrough, and when subjected to a force exceeding the first friction force, the elastic sealing member moves relative to the flexible electrode along the length direction of the flexible electrode. The third through channel of the elastic sealing member is configured to apply a second friction force to the traction member penetrating therethrough. When subjected to a force exceeding the second friction force, the elastic sealing member moves relative to the traction member along a length direction of the traction member.

In some embodiments, a difference between the first friction force and the second friction force is within a preset range, and the force of the first threshold is configured such that, upon being subjected to the force, the elastic sealing member can overcome the first friction force and the second friction force and move synchronously relative to the flexible electrode and the traction member.

In some embodiments, the auxiliary fixing assembly further comprises a second fixing member, which is connected to the first fixing member in a movable manner, and an elastic sealing member is provided between the first fixing member and the second fixing member. The second fixing member serves to move toward the first fixing member to apply a force to the elastic sealing member to press it against the first fixing member.

In some embodiments, the first fixing member and the second fixing member are used to cooperate to apply a compressive force exceeding a second threshold to the elastic sealing member, so that the elastic sealing member can deform to compress the flexible electrode.

In some embodiments, the first fixing member and the second fixing member are in a snap-fit connection, and/or the first fixing member and the second fixing member are in a locking connection.

In some embodiments, one of the first fixing member and the second fixing member is provided with a recessed portion, and the other is provided with a protruding portion that matches the recessed portion, and the protruding portion is snapped into the recessed portion to achieve the snap-fit connection between the first fixing member and the second fixing member.

In some embodiments, the elastic sealing member has a second through channel through which the flexible electrode penetrates, and the second fixing member has a fourth through channel through which the flexible electrode penetrates. The first through channel, the second through channel, and the fourth through channel remain aligned along the length direction of the flexible electrode during the connection between the second fixing member and the first fixing member.

In some embodiments, the flexible electrode comprises a first insulating layer, a first conductive layer, and a second insulating layer stacked along a thickness direction of the flexible electrode. The electrode site is formed on the first conductive layer, and the first insulating layer and/or the second insulating layer is not provided outside a portion of the first conductive layer corresponding to the electrode site.

In some embodiments, a traction portion is formed at a distal end of the traction member, and the traction portion acts on the auxiliary implantation portion. The traction member is used to detach the traction portion from the auxiliary implantation portion when subjected to a force applied toward the proximal contact portion of the flexible electrode.

Compared with the prior art, the beneficial effects of the embodiments of the present application are as follows: the present application utilizes a synergistic action of an elastic sealing member and an auxiliary fixing assembly, wherein the elastic sealing member can abut against the auxiliary fixing assembly after the distal electrode portion of the flexible electrode is implanted into the target tissue to a preset depth, so that the elastic sealing member can apply a compressive force to the flexible electrode at the preset depth, thereby achieving the purpose of keeping the distal electrode portion of the flexible electrode at the preset depth. This effectively limits the implantation depth of the flexible electrode, improves the implantation stability of the flexible electrode, and avoids problems such as excessive implantation depth or deviation from the target position of the flexible electrode. Furthermore, the elastic sealing member is deformed upon abutting against the auxiliary fixing assembly to achieve a sealed connection between the elastic sealing member and the auxiliary fixing assembly, thereby enabling sealing of the wound where flexible electrode is implanted, reducing the risk of leakage or contamination of body fluids (such as blood, cerebrospinal fluid, etc.) or gases, and providing important technical support for the precise implantation and long-term stable use of the flexible electrode.

### BRIEF DESCRIPTION OF THE DRAWINGS

In drawings that are not necessarily drawn to scale, the same reference numerals may describe similar parts in different views. The drawings generally show various embodiments by way of example rather than limitation, and are used together with the description and the claims to describe the disclosed embodiments. Appropriately, the same reference signs are used throughout the drawings to refer to the same or similar parts. Such embodiments are illustrative, and are not intended to be exhaustive or exclusive embodiments of the present device or method.

FIG. 1 is a schematic diagram of the three-dimensional structure of a flexible neural electrode assembly according to an embodiment of the present application.

FIG. 2 is a schematic diagram of the installation process of the flexible neural electrode assembly according to the embodiment of the present application.

FIG. 3 is a schematic diagram of the installation process of the flexible neural electrode assembly during the implantation process according to the embodiment of the present application.

FIG. 4 is a schematic diagram of the installation process of the flexible neural electrode assembly implanted into the brain according to the embodiment of the present application.

The members indicated by the reference numerals in the figures:
1. Flexible electrode; 11. Proximal contact portion; 12. Lead connection portion; 13. Distal electrode portion; 14. Auxiliary implantation portion; 2. Traction member; 21. Traction portion; 3. Auxiliary fixing assembly; 31. First fixing member; 32. Second fixing member; 33. Recessed portion; 34. Protruding portion; 4. Elastic sealing member.

### DETAILED DESCRIPTION

In order to enable those skilled in the art to better understand the technical solution of the present application, the present application will be described in detail with reference to the drawings and specific embodiments. The embodiments of the present application will be described in further detail below with reference to the drawings and specific embodiments, but not as a limitation of the present application.

The terms “first”, “second”, and similar words used in the present application do not indicate any order, quantity, or importance, but are only used to distinguish different parts. Similar words such as “comprising” or “containing” mean that the elements before the word cover the elements listed after the word, and the possibility of covering other elements is not excluded. “Up”, “Down”, “Left”, and “Right” are only used to indicate the relative position relationship. When the absolute position of the described object changes, the relative position relationship may further change accordingly.

In the present application, when it is described that a specific device is located between a first device and a second device, there may or may not be an intervening device between the specific device and the first device or the second device. When it is described that a specific device is connected to another device, the specific device may be directly connected to the other device without an intervening device, or may not be directly connected to the other device but with an intervening device.

All terms (including technical terms or scientific terms) used in the present application have the same meanings as those understood by ordinary technicians in the field to which the present application belongs, unless otherwise defined. It should further be understood that terms defined in, for example, general dictionaries should be interpreted as having meanings consistent with their meanings in the context of the related art, and should not be interpreted in an idealized or extremely formal sense unless explicitly defined here.

Techniques, methods, and equipment known to those skilled in the related art may not be discussed in detail, but they should be regarded as part of the specification under appropriate circumstances.

In the present application, the term “proximal” is intended to refer to the end close to the operator (such as a doctor) performing the implantation operation, while the term “distal” is intended to refer to the side close to the target site of the implanted neural signaling device to be implanted.

All terms (including technical terms or scientific terms) used in the present application have the same meaning as understood by ordinary skilled persons in the art to which the present application belongs, unless otherwise specifically defined. It should further be understood that terms defined in general dictionaries should be interpreted as having meanings consistent with their meanings in the context of the relevant art, and should not be interpreted in an idealized or overly formal sense unless explicitly defined in this way herein. The techniques, methods, and equipment known to those ordinary skilled in the relevant field may not be discussed in detail, but in appropriate circumstances, such techniques, methods, and equipment should be considered as part of the specification.

The embodiment of the present application provides a flexible neural electrode assembly, which can be used in the medical field, etc. A detailed introduction to a flexible neural electrode assembly according to the embodiment of the present application will be described in detail in the following text in conjunction with the accompanying drawings.

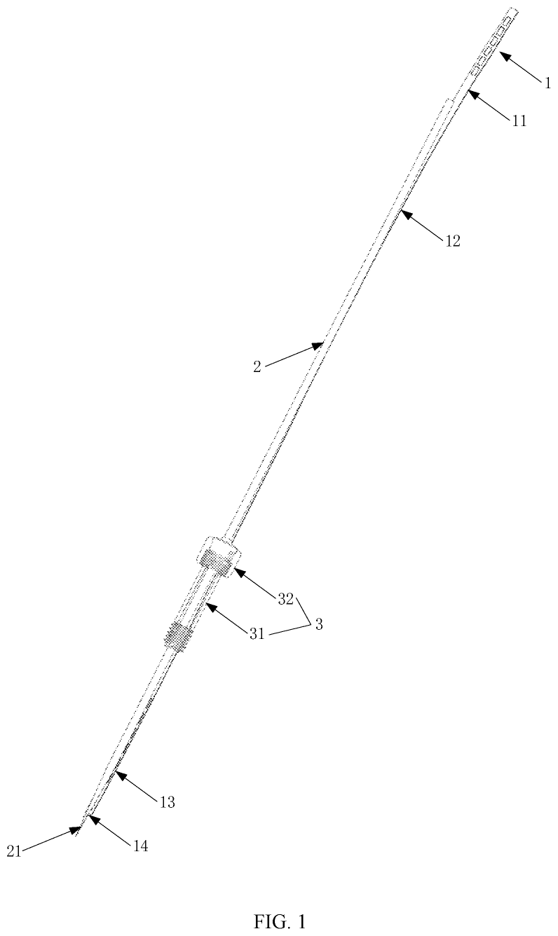

As shown in FIGS. 1 to 4, the flexible neural electrode assembly mentioned above comprises a flexible electrode 1, a traction member 2, an auxiliary fixing assembly 3, and an elastic sealing member 4. Flexible electrode 1 is made of a flexible material and includes a proximal contact portion 11, a lead connection portion 12, a distal electrode portion 13, and an auxiliary implantation portion 14 sequentially connected along the length direction of flexible electrode 1. The distal electrode portion 13 and the auxiliary implantation portion 14 are used for implantation into a target tissue, and the distal electrode portion 13 includes at least one electrode site for applying electrical stimulation to the target tissue and/or acquiring potential signals of the target tissue. The traction member 2 acts on the auxiliary implantation portion 14 to implant the flexible electrode 1 toward the target area via the auxiliary implantation portion 14. The auxiliary fixing assembly 3 is connected to the surface of the target tissue and/or the accessory anatomical structure of the target tissue. The flexible electrode 1 and the traction member 2 both penetrate through the auxiliary fixing assembly 3 for implantation into the target tissue. The elastic sealing member 4 is sleeved outside the flexible electrode 1 and the traction member 2. The elastic sealing member 4 is configured to abut against the auxiliary fixing assembly 3 at least after the distal electrode portion 13 is implanted into the target tissue to a preset depth, and deform under the action of the auxiliary fixing assembly 3 to compress the flexible electrode 1, so that the distal electrode portion 13 of the flexible electrode 1 is fixed at the preset depth, and the flexible electrode 1 is sealingly connected to the auxiliary fixing assembly 3 via the elastic sealing member 4.

The flexible materials used for the flexible electrode 1 mentioned above can exhibit good conductivity and biocompatibility, which can ensure the stable operation of the flexible electrode 1 for a long time. It further has a certain degree of flexibility, such that the flexible electrode 1 can deform with the movement of human tissue after implantation therein. Of course, the flexible electrode 1 mentioned above can be made of metal materials, non-metal materials, or a combination of non-metal and metal materials. The materials used for the flexible electrode 1 are not specifically limited in the present application.

The flexible electrode 1 mentioned above can be in the shape of an elongated strip-like structure, with the proximal contact portion 11, the lead connection portion 12, the distal electrode portion 13, and the auxiliary implantation portion 14 connected sequentially along the length direction of the flexible electrode 1. Among them, the proximal contact portion 11, the lead connection portion 12, and the distal electrode portion 13 are all in the shape of an elongated strip-like structure, and the auxiliary implantation portion 14 can be constructed as an aperture structure or a groove structure.

The auxiliary implantation portion 14, which is constructed as an aperture structure, may have a diameter ranging from 0.002 mm to 2 mm. Under the action of the traction member 2, the auxiliary implantation portion 14 can drive the flexible electrode 1 to be precisely implanted into the target tissue.

The electrode sites of the aforementioned distal electrode portion 13 can be electrically connected to the proximal contact portion 11 through the lead connection portion 12. The number of the electrode sites of the distal electrode portion 13 ranges from 1 to 200, wherein the number of the electrode sites for acquiring potential signals of the target tissue can range from 1 to 2000. These electrode sites can be used to apply electrical stimulation to the target tissue or to acquire potential signals of the target tissue. The design of the number of the electrode sites mentioned above fully considers the coverage range of electrical stimulation and the resolution of recorded signals, ensuring their functionality in various application scenarios.

The width of the proximal contact portion 11 mentioned above can range from 100 um to 10 mm, with its thickness controlled below 0.2 mm. It can achieve signal interaction with an external apparatus through an electrical connection.

The width of the aforementioned lead connection portion 12 may vary at different positions, and its structural design is specifically a stacked design, that is, formed by stacking multiple layers.

During the implantation process, the aforementioned traction member 2 is used to guide the distal electrode portion 13 of the flexible electrode 1 to a preset depth in the target tissue by applying a force toward the distal end. After the distal electrode portion 13 of the flexible electrode 1 reaches the preset depth in the target tissue, the traction member 2 separates from the auxiliary implantation portion 14 of the flexible electrode 1, and the traction member 2 is removed from the target tissue, so that the distal electrode portion 13 of the flexible electrode 1 is positioned at the preset depth in the target tissue.

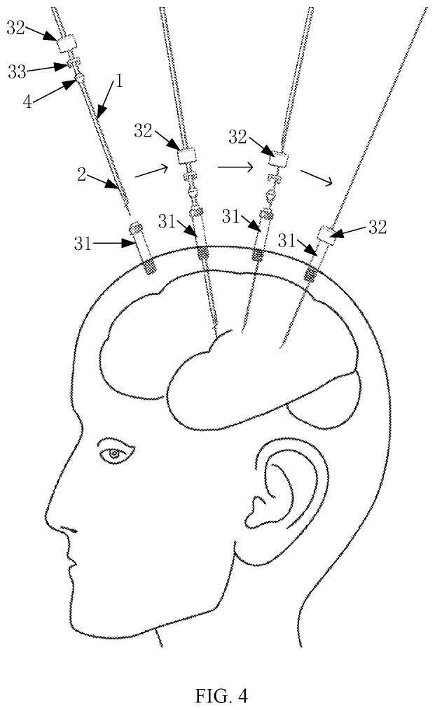

The surface of the aforementioned target tissue and/or accessory anatomical structure of the target tissue can be interpreted as biological structures located outside and close to the target tissue. Specifically, as shown in FIG. 4, in the case where the target tissue is brain tissue, the accessory anatomical structure of the target tissue can be interpreted as the skull.

The aforementioned auxiliary fixing assembly 3 can be made of biocompatible materials, such as polyetheretherketone (PEEK), titanium, titanium alloy, stainless steel, or other medical-grade polymer materials, to ensure the stability and compatibility with tissues of the fixation portion during long-term use.

The aforementioned elastic sealing member 4 can be made of materials with high elasticity, high flexibility, and biocompatibility, such as silicone and rubber, so that the elastic sealing member 4 can undergo moderate deformation when subjected to the compressive force applied by the auxiliary fixing assembly 3.

It can be understood that the deformed elastic sealing member 4 can not only tightly fit with the auxiliary fixing assembly 3, but also deform and compress the flexible electrode 1, thereby the flexible electrode 1 can be sealingly connected to the auxiliary fixing assembly 3 via the elastic sealing member 4, ensuring that the wound where the flexible electrode 1 is implanted can be sealed. After sealing, not only the leakage of body fluids (such as blood, cerebrospinal fluid, etc.) or gases can be prevented, but also external contaminants can be effectively prevented from entering the body. Moreover, the elastic sealing member 4 can further fix the distal electrode portion 13 of the flexible electrode 1 at the preset depth in the target tissue by compressing the flexible electrode 1, thereby achieving precise limitation of the implantation depth of the flexible electrode 1 and ensuring its safety during long-term use.

The length of the cross-section of the elastic sealing member 4 mentioned above can range from 0.2 mm to 200 mm, and its width can range from 0.1 mm to 100 mm.

The size and shape of the aforementioned elastic sealing member 4 can be adjusted depending on the implantation requirements of the flexible electrode 1, such that the elastic sealing member 4 can apply an appropriate compressive force to the flexible electrode 1 after deformation under force. In this manner, the implantation depth of the flexible electrode 1 is limited while ensuring that it is not damaged.

With the synergistic action of the elastic sealing member 4 and the auxiliary fixing assembly 3, the present application achieves effective limitation of the implantation depth of the flexible electrode 1, improves the implantation stability of the flexible electrode 1, and avoids problems such as excessive implantation depth or deviation from the target position of the flexible electrode 1. Wherein, the elastic sealing member 4 can abut against the auxiliary fixing assembly 3 after the distal electrode portion 13 of the flexible electrode 1 is implanted into the target tissue to a preset depth, so that the elastic sealing member 4 can apply a compressive force to the flexible electrode 1 at the preset depth, thereby achieving the purpose of keeping the distal electrode portion 13 of the flexible electrode 1 at the preset depth. Furthermore, the elastic sealing member 4 can deform upon abutting against the auxiliary fixing assembly 3 to achieve a sealed connection between the elastic sealing member 4 and the auxiliary fixing assembly 3, thereby enabling sealing of the wound where the flexible electrode 1 is implanted, reducing the risk of leakage or contamination of body fluids (such as blood, cerebrospinal fluid, etc.) or gases, and providing important technical support for the precise implantation and long-term stable use of the flexible electrode 1.

In some embodiments, as shown in FIGS. 1 to 4, the auxiliary fixing assembly 3 includes a first fixing member 31 connected to the surface of the target tissue and/or the accessory anatomical structure of the target tissue. The first fixing member 31 is provided with a first through channel, through which the flexible electrode 1 and the traction member 2 are implanted into the target tissue. The elastic sealing member 4 abuts against the proximal end of the first fixing member 31.

In this way, the first fixing member 31 can be fixed to the surface of the target tissue and/or the accessory anatomical structure of the target tissue, which facilitates the followed precise implantation of the flexible electrode 1. Once being fixed in position, the first fixing member 31 abuts against the elastic sealing member 4, which can apply a stable force to the elastic sealing member 4 and improve the structural stability.

The first fixing member 31 mentioned above can be connected to the surface of the target tissue and/or the accessory anatomical structure of the target tissue in a detachable manner, such as a threaded connection, a snap-fit connection, etc. Of course, it can further be connected to the surface of the target tissue and/or the accessory anatomical structure of the target tissue using adhesive bonding. The present application does not impose specific limitations thereon, as long as the first fixing member 31 can be stably installed.

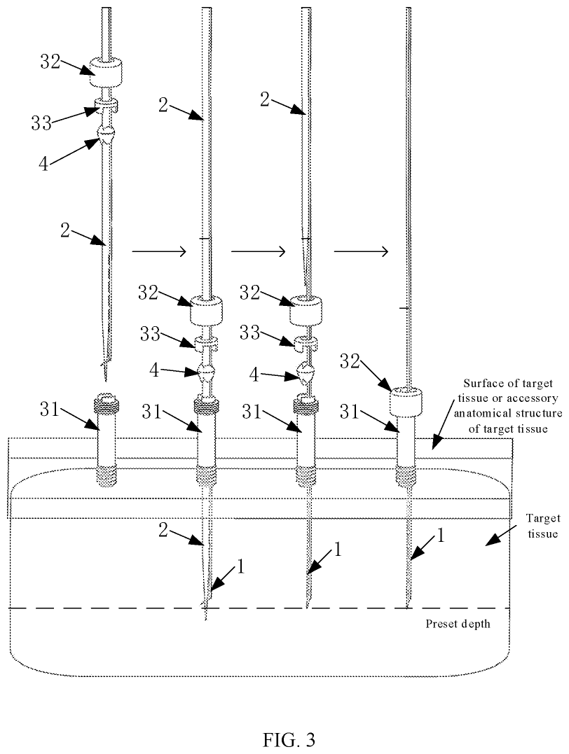

For example, as shown in FIGS. 3 and 4, the distal end of the first fixing member 31 may be formed with a threaded structure so as to be connected to the surface of the target tissue and/or the accessory anatomical structure of the target tissue through the threaded structure.

The cross-sectional dimension of the aforementioned first fixing member 31 ranges from 0.1 mm to 20 mm, and its length ranges from 0.2 mm to 40 mm, providing sufficient support area for the entire flexible neural electrode assembly.

The cross-sectional dimension of the first through channel mentioned above ranges from 0.005 mm to 10 mm, so as to match the lead connection portion 12 of the flexible electrode 1 and the traction member 2, ensuring that the flexible electrode 1 can be smoothly implanted while the implantation path of the flexible electrode 1 can be precise without deviation.

The shape of the distal end of the elastic sealing member 4 mentioned above can be adapted to the shape of the proximal end of the first fixing member 31, so that the elastic sealing member 4 and the first fixing member 31 can stably abut against each other, and the contact sealing performance between the elastic sealing member 4 and the first fixing member 31 can be ensured.

For example, the proximal end of the first fixing member 31 may be formed with a first groove, and the distal end of the elastic sealing member 4 may be at least partially embedded in the first groove to ensure the stability of the connection between the elastic sealing member 4 and the first fixing member 31.

In some embodiments, the elastic sealing member 4 has a second through channel through which the flexible electrode 1 penetrates, and the second through channel is configured to deform toward the flexible electrode 1 under the action of the auxiliary fixing assembly 3, so as to abut against the flexible electrode 1.

In this way, after the second through channel is deformed under the force applied by the first fixing member 31, a relatively appropriate force can be applied to the flexible electrode 1 through the deformed second through channel. This not only enables the stable implantation of the flexible electrode 1 to be guided via the second through channel, but also allows the deformed second through channel to abut against the flexible electrode 1, thereby achieving a better sealing effect.

The cross-sectional dimension of the second through channel mentioned above ranges from 0.005 mm to 50 mm.

The second through channel mentioned above is configured to allow the flexible electrode 1 to pass through smoothly, meanwhile to abut against the flexible electrode 1 after deformation, and thus form a sealing connection with the flexible electrode 1. Therefore, the size of the second through channel should not be too large, it is sufficient to form a certain gap between it and the flexible electrode 1.

The shape of the second through channel mentioned above can be adapted to the shape of the flexible electrode 1. For example, when the flexible electrode 1 is sheet-shaped, the shape of the second through channel can be rectangular.

The aforementioned second through channel abutting against the flexible electrode 1 can be interpreted as the inner wall of the second through channel wrapping around the flexible electrode 1 and applying appropriate force to the flexible electrode 1 to ensure the sealing effect.

In some embodiments, the elastic sealing member 4 further has a third through channel through which the traction member 2 penetrates, and the second through channel and the third through channel are in communication with each other or separately arranged.

In the case where the second through channel and the third through channel are separately arranged, the passages through which the flexible electrode 1 and the traction member 2 penetrate can be made to not affect each other, to ensure the accuracy of the implantation of the flexible electrode 1 during the implantation process, thereby avoiding wear or misalignment of the flexible electrode 1 and the traction member 2 caused by mechanical deviations, and ensuring the sealing performance between the second through channel and the flexible electrode 1 through a separate passage design.

In the aforementioned case where the second through channel and the third through channel are in communication with each other, it can facilitate the manufacturing and processing of the elastic sealing member 4, and it can further facilitate the penetration of the flexible electrode 1 and the traction member 2, improving operational convenience.

The length direction of the second through channel and the length direction of the third through channel mentioned above can be parallel to each other, which is beneficial for maintaining the overall symmetry of the elastic sealing member 4 and ensures its stable performance during sealing and limiting.

In some embodiments, the elastic sealing member 4 is configured to move relative to the flexible electrode 1 along the length direction of the flexible electrode 1 when subjected to a force exceeding a first threshold.

In this way, by means of the elastic sealing member 4 that can move relative to the flexible electrode 1, the implantation depth of the flexible electrode 1 can be flexibly adjusted. After the distal electrode portion 13 is implanted to the preset depth in the target tissue, the elastic sealing member 4 then abuts against the auxiliary fixing assembly 3 to ensure the precise positioning of the flexible electrode 1.

It is appreciated that the aforementioned elastic sealing member 4 is configured such that it cannot move relative to the flexible electrode 1 along the length direction of the flexible electrode 1 when subjected to a force not exceeding the first threshold. At this time, the relative position between the elastic sealing member 4 and the flexible electrode 1 remains unchanged. That is, the elastic sealing member 4 can approach the first fixing member 31 with the implantation of the flexible electrode 1.

In some embodiments, the second through channel and/or the third through channel is slit-shaped. In this way, by ensuring smooth passage of the flexible electrode 1 through the slit-shaped second through channel and/or third through channel, the sealing effect of the flexible electrode 1 can be achieved through the slit-shaped structure, eliminating the problem of ineffective sealing of the flexible electrode 1 due to an excessively large gap between the second through channel and the flexible electrode 1. It can further prevent the flexible electrode 1 from being worn due to vibration or movement during the implantation process.

The cross-sectional shape of the aforementioned slit-shaped second through channel and/or third through channel can be one or a combination of the following: zigzag, linear, and elliptical. Among them, the linear second and/or third through channels can serve as linear guidance, while the zigzag and elliptical second through channel and/or third through channel can better accommodate the complex motion requirements of the flexible electrode 1 and enhance its fixation effect with the flexible electrode 1.

The third through channel mentioned above can be adapted to the traction member 2 in shape and size. For example, the shape of the third through channel is an aperture.

In some embodiments, the second through channel of the elastic sealing member 4 is configured to apply a first friction force to the flexible electrode 1 penetrating therethrough. When the force applied to the elastic sealing member 4exceeds a first friction force, the elastic sealing member 4 moves relative to the flexible electrode 1 along the length direction of the flexible electrode 1. The third through channel of the elastic sealing member 4 is configured to apply a second friction force to the traction member 2 penetrating therethrough. When the force applied to the elastic sealing member 4 exceeds a second friction force, the elastic sealing member 4 moves relative to the traction member 2 along the length direction of the traction member 2.

In this way, with the design of the first friction force and second friction force, the elastic sealing member 4 can only move relative to the flexible electrode 1 and the traction member 2 after being subjected to a force reaching a certain threshold. This improves the stability of the interaction between the elastic sealing member 4 and the flexible electrode 1, and the traction member 2, and achieves the objective of precise implantation of the flexible electrode 1 when driven by the traction member 2.

It can be appreciated that when the force on the elastic sealing member 4 does not exceed the first friction force, the elastic sealing member 4 remains stationary relative to the flexible electrode 1. When the force on the elastic sealing member 4 does not exceed the second friction force, the elastic sealing member 4 remains stationary relative to the traction member 2.

The aforementioned application of the first friction force by the second through channel of the elastic sealing member 4 to the flexible electrode 1 passing therethrough can be interpreted as being achieved based on the parameter design of the second through channel, including, but not limited to, the material design, size design, and shape design of the second through channel.

The aforementioned application of the second friction force by the third through channel of the elastic sealing member 4 to the traction member 2 passing therethrough can be interpreted as being achieved based on the parameter design of the third through channel, including, but not limited to, the material design, size design, and shape design of the second through channel.

In some embodiments, the difference between the first friction force and the second friction force is within a preset range, and the force of the first threshold is configured such that, upon being subjected to this force, the elastic sealing member 4 can overcome the first friction force and the second friction force and move synchronously relative to the flexible electrode 1 and the traction member 2.

In this way, the elastic sealing member 4 can overcome the first friction force and the second friction force after being subjected to a force exceeding the first threshold, and slide on the flexible electrode 1 and the traction member 2 simultaneously. This avoids problems such as uncoordinated movement or even pulling of the elastic sealing member 4 caused by an excessive friction force between the elastic sealing member 4 and either the flexible electrode 1 or the traction member 2.

It can be understood that the difference between the aforementioned first friction force and the second friction force should not be excessively large. The preset range can enable the elastic sealing member 4 to slide synchronously and smoothly relative to the flexible electrode 1 and the traction member 2 after being subjected to a force.

Specifically, one of the first friction force and the second friction force may be equal to the force of the first threshold, while the other may be less than or equal to the force of the first threshold.

In some embodiments, as shown in FIGS. 1 to 4, the auxiliary fixing assembly 3 further comprises a second fixing member 32, which is connected to the first fixing member 31 in a movable manner. The elastic sealing member 4 is provided between the first fixing member 31 and the second fixing member 32, and the second fixing member 32 serves to move toward the first fixing member 31 to apply a force to the elastic sealing member 4 to press it against the first fixing member 31.

In this way, by moving the second fixing member 32 toward the direction close to the first fixing member 31, the elastic sealing member 4 can be continuously compressed and gradually apply a force to the flexible electrode 1, ensuring the stability of the force applied by the auxiliary fixing assembly 3 to the elastic sealing member 4.

The shape of the proximal end of the elastic sealing member 4 mentioned above can be adapted to the shape of the distal end of the second fixing member 32, so that the elastic sealing member 4 and the second fixing member 32 can stably abut against each other, and the contact sealing performance between the elastic sealing member 4 and the second fixing member 32 can be ensured.

For example, the distal end of the second fixing member 32 may be formed with a second groove, and the proximal end of the elastic sealing member 4 may be at least partially embedded in the second groove to ensure the stability of the connection between the elastic sealing member 4 and the second fixing member 32.

The cross-sectional dimension of the abovementioned second fixing member 32 ranges from 0.1 mm to 200 mm, and its length ranges from 0.2 mm to 40 mm, allowing the second fixing member 32 to connect stably to the first fixing member 31.

In some embodiments, the elastic sealing member 4 has a second through channel through which the flexible electrode 1 penetrates, and the second fixing member 32 has a fourth through channel through which the flexible electrode 1 penetrates. The first through channel, the second through channel, and the fourth through channel remain aligned along the length direction of the flexible electrode 1 during the connection process between the second fixing member 32 and the first fixing member 31, in order to avoid compression or damage to the flexible electrode 1 due to misalignment of the first through channel, the second through channel, and the fourth through channel during the connection between the second fixing member 32 and the first fixing member 31, thereby improving the implantation accuracy and service life of the flexible electrode 1.

The fourth through channel mentioned above can further be used for the passage of the traction member 2, such that the traction member 2 and the flexible electrode 1 can share the fourth through channel, achieving a compact structural design. Of course, the fourth through channel may not be further used for the passage of the traction member 2, and the second fixing member 32 can further have a fifth through channel through which the traction member 2 penetrates.

In some embodiments, as shown in FIGS. 1 to 4, the first fixing member 31 and the second fixing member 32 are used to cooperate to apply a compressive force exceeding a second threshold to the elastic sealing member 4, thereby enabling the elastic sealing member 4 to deform so as to compress the flexible electrode 1.

In this way, during the connection between the first fixing member 31 and the second fixing member 32, by applying a compressive force exceeding the second threshold to the elastic sealing member 4, the elastic sealing member 4 can undergo a deformation sufficient to compress the flexible electrode 1, thereby wrapping around the outside of the flexible electrode 1 and enabling a sealed connection between the elastic sealing member 4 and the flexible electrode 1.

In some embodiments, the first fixing member 31 and the second fixing member 32 are in a snap-fit connection, and/or the first fixing member 31 and the second fixing member 32 are in a locking connection. The aforementioned connection method between the first fixing member 31 and the second fixing member 32 is stable, which can effectively ensure the implantation stability of the flexible electrode 1.

It can be understood that the first fixing member 31 and the second fixing member 32 under the snap-fit connection may not be rotatable relative to each other, while the first fixing member 31 and the second fixing member 32 under the locking connection need to generate an axial fastening force through relative rotation.

In the case where the connection method between the first fixing member 31 and the second fixing member 32 includes a snap-fit connection and a locking connection, the snap-fit connection and the locking connection may occur sequentially or simultaneously. For example, the first fixing member 31 and the second fixing member 32 are first engaged in a snap-fit connection and then secured via a locking connection.

In a case where a connection method between the first fixing member 31 and the second fixing member 32 includes a snap-fit connection and a locking connection, the first fixing member 31 may be provided with a snap-fit connection portion and a locking connection portion, which may be integrally formed or separately provided. Correspondingly, the second fixing member 32 may be provided with a snap-fit connection portion and a locking connection portion, which may be integrally formed or separately provided. The present application does not specifically limit this. If the integrated design is adopted, the number of parts can be reduced, and installation efficiency can be improved, which is suitable for implantation scenarios that require fast operation and a compact structure. If a separately and independently provided design is adopted, it can provide greater flexibility for the structural design of the first fixing member 31 and the second fixing member 32, which is suitable for complex implantation scenarios and situations that require separate adjustment and replacement of the first fixing member 31 and the second fixing member 32.

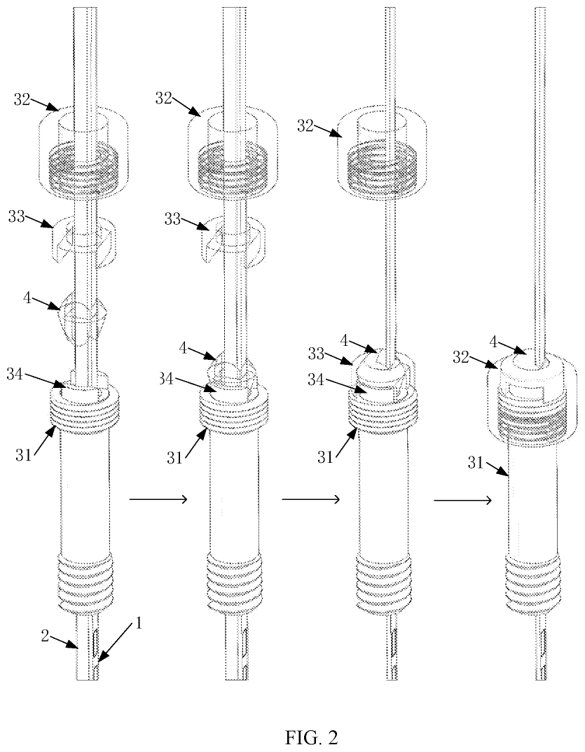

As shown in FIGS. 2 to 4, in the case where the first fixing member 31 and the second fixing member 32 are in the snap-fit connection, one of the first fixing member 31 and the second fixing member 32 is provided with a recessed portion 33, and the other is provided with a protruding portion 34 that matches the recessed portion 33. The protruding portion 34 is snapped into the recessed portion 33 to achieve the snap-fit connection between the first fixing member 31 and the second fixing member 32.

As shown in FIGS. 2 to 4, in the case where the first fixing member 31 and the second fixing member 32 are in the locking connection, the first fixing member 31 may have a first locking structure, the second fixing member 32 may have a second locking structure, and the first locking structure and the second locking structure may be in the locking connection. For example, the first locking structure and the second locking structure can be in threaded connection to generate an axial fastening force by rotating the second fixing member 32 relative to the first fixing member 31, thereby stabilizing the overall structure of the auxiliary fixing assembly 3.

When only a locking connection is adopted to connect the first fixing member 31 and the second fixing member 32, the elastic sealing member 4 cannot rotate relative to the first fixing member 31 under the restriction of the first fixing member 31, in order to avoid misalignment of the first through channel, the second through channel, and the fourth through channel during the installation process of the first fixing member 31 and the second fixing member 32.

Specifically, referring to FIG. 2, which illustrates the installation process of the flexible neural electrode assembly. The first figure from the left shows the separation of the elastic sealing member 4, the first fixing member 31, and the second fixing member 32. The second figure from the left shows that the lower part of the elastic sealing member 4 abuts against the first fixing member 31. The third figure from the left shows that the protruding portion 34 of the first fixing member 31 is snapped into the recessed portion 33 of the second fixing member 32, thereby achieving the snap-fit connection between the first fixing member 31 and the second fixing member 32, at which point the elastic sealing member 4 is compressed between the first fixing member 31 and the second fixing member 32. The fourth figure from the left shows that the first fixing member 31 and the second fixing member 32 are engaged in the locking connection, thereby completing the restriction to the flexible electrode 1 and enabling the flexible electrode 1 to be sealingly connected to the auxiliary fixing assembly 3 via the elastic sealing member 4.

With further reference to FIGS. 3 and 4, FIGS. 3 and 4 both illustrate the installation process of the flexible neural electrode assembly. The flexible neural electrode assembly shown from left to right is in a first state, a second state, a third state, and a fourth state, respectively. In the first state, the first fixing member 31 is fixed to the surface of the target tissue and/or the accessory anatomical structure of the target tissue. In the second state, the flexible electrode 1, driven by the traction member 2, is implanted into the target tissue to a preset depth through the first through channel of the first fixing member 31. In the third state, the traction member 2 is separated from the flexible electrode 1 and subsequently removed. In the fourth state, the first fixing member 31 and the second fixing member 32 are in a snap-fit connection and a locking connection to complete the restriction to the flexible electrode 1 and enable the flexible electrode 1 to be sealingly connected with the auxiliary fixing assembly 3 via the elastic sealing member 4.

Through the synergistic action of the aforementioned auxiliary fixing assembly 3 and elastic sealing member 4, there are at least the following four implantation methods for the flexible electrode 1.

The first implantation method: In the case where one connection method selected from a snap-fit connection and a locking connection is adopted between the first fixing member 31 and the second fixing member 32, the first fixing member 31 of the auxiliary fixing assembly 3 is first fixed to the surface of the target tissue and/or the accessory anatomical structure of the target tissue to form an implantation channel for the flexible electrode 1. Subsequently, the distal electrode portion 13 of the flexible electrode 1 and the traction member 2 are implanted into the target tissue through the first through channel of the first fixing member 31 until the distal electrode portion 13 reaches the preset depth in the target tissue. By means of a force applied toward the proximal end, the traction member 2 is separated from the auxiliary implantation portion 14 of the flexible electrode 1 and is removed, leaving the distal electrode portion 13 of the flexible electrode 1 inside the target tissue, while the lead connection portion 12 and the proximal contact portion 11 remain outside the target tissue. The elastic sealing member 4 is moved along the lead connection portion 12 of the flexible electrode 1 to the proximal end of the first fixing member 31. Next, the second fixing member 32 of the auxiliary fixing assembly 3 is snap-fitted with its first fixing member 31 to form a firm mechanical fixation. During the snap-fitting process, the elastic sealing member 4 is located between the first fixing member 31 and the second fixing member 32, and is subjected to sufficient compressive force under the action of the second fixing member 32, so as to tightly fit with the flexible electrode 1 and the auxiliary fixing assembly 3, thereby achieving the fixation of the flexible electrode 1 and the sealing of the wound.

The second mode of synergistic action: in the case where one connection method selected from a snap-fit connection and a locking connection is adopted between the first fixing member 31 and the second fixing member 32, and the elastic sealing member 4 is used to control the implantation depth of the flexible electrode 1, the first fixing member 31 of the auxiliary fixing assembly 3 is first fixed on the surface of the target tissue and/or the accessory anatomical structure of the target tissue to form the implantation channel for the flexible electrode 1. The elastic sealing member 4 is moved along the lead connection portion 12 of the flexible electrode 1, so that the distal electrode portion 13 of the flexible electrode 1 is located at the preset depth in the target tissue after implantation. Subsequently, the distal electrode portion 13 of the flexible electrode 1 and the traction member 2 are implanted into the target tissue through the first through channel of the first fixing member 31 until the elastic sealing member 4 reaches the proximal end of the first fixing member 31, at which point the distal electrode portion 13 of the flexible electrode 1 reaches the preset depth in the target tissue. By means of a force applied toward the proximal end, the traction member 2 is separated from the auxiliary implantation portion 14 of the flexible electrode 1 and is removed, leaving the distal electrode portion 13 of the flexible electrode 1 inside the target tissue, while the lead connection portion 12 and the proximal contact portion 11 remain outside the target tissue. Next, the second fixing member 32 of the auxiliary fixing assembly 3 is snap-fitted with its first fixing member 31 to form a firm mechanical fixation. During snap-fitting process, the elastic sealing member 4 is located between the first fixing member 31 and the second fixing member 32, and is subjected to sufficient compressive force under the action of the second fixing member 32 to tightly fit with the flexible electrode 1 and the auxiliary fixing assembly 3, thereby achieving the fixation of the flexible electrode 1 and the sealing of the wound.

The third mode of synergistic action: In the case where a snap-fit connection and a locking connection are adopted between the first fixing member 31 and the second fixing member 32, the first fixing member 31 of the auxiliary fixing assembly 3 is first fixed to the surface of the target tissue and/or the accessory anatomical structure of the target tissue, forming an implantation channel for the flexible electrode 1. Subsequently, the distal electrode portion 13 of the flexible electrode 1 and the traction member 2 are implanted into the target tissue through the first through channel of the first fixing member 31 until the distal electrode portion 13 reaches the preset depth in the target tissue. By means of a force applied toward the proximal end, the traction member 2 is separated from the auxiliary implantation portion 14 of the flexible electrode 1 and is removed, so that the distal electrode portion 13 of the flexible electrode 1 is fixed inside the target tissue, and the lead connection portion 12 and the proximal contact portion 11 remain outside the target tissue. After the traction member 2 is removed, the elastic sealing member 4 is moved along the lead connection portion 12 of the flexible electrode 1 to the proximal end of the first fixing member 31. The second fixing member 32 is moved and fixed to the proximal end of the elastic sealing member 4. Next, the second fixing member 32 of the auxiliary fixing assembly 3 is snap-fitted with its first fixing member 31 to ensure that the elastic sealing member 4 and the lead connection portion 12 are not damaged due to torsional forces in subsequent operations. Subsequently, the second fixing member 32 is further in a locking connection with the first fixing member 31 to continue acting on the elastic sealing member 4, causing the elastic sealing member 4 to be subjected to a compressive force exceeding the second threshold and tightly fit with the first fixing member 31 and the flexible electrode 1, thereby achieving fixation of the flexible electrode 1 and sealing of the wound.

The fourth mode of synergistic action: In the case where a snap-fit connection and a locking connection are adopted between the first fixing member 31 and the second fixing member 32, and the elastic sealing member 4 is used to control the implantation depth of the flexible electrode 1, the first fixing member 31 of the auxiliary fixing assembly 3 is first fixed to the surface of the target tissue and/or the accessory anatomical structure of the target tissue, forming an implantation channel for the flexible electrode 1. The elastic sealing member 4 is moved along the lead connection portion 12 of the flexible electrode 1, so that the distal electrode portion 13 of the flexible electrode 1 is located at the preset depth in the target tissue after implantation. Subsequently, the distal electrode portion 13 of the flexible electrode 1 and the traction member 2 are implanted into the target tissue through the first through channel of the first fixing member 31 until the elastic sealing member 4 reaches the proximal end of the first fixing member 31 and the distal electrode portion 13 of the flexible electrode 1 reaches the preset depth in the target tissue. By means of a force applied toward the proximal end, the traction member 2 is separated from the auxiliary implantation portion 14 of the flexible electrode 1 and is removed, so that the distal electrode portion 13 of the flexible electrode 1 is fixed inside the target tissue, and the lead connection portion 12 and the proximal contact portion 11 remain outside the target tissue. The second fixing member 32 is moved and fixed to the proximal end of the elastic sealing member 4. Next, the second fixing member 32 of the auxiliary fixing assembly 3 is snap-fitted with its first fixing member 31 to ensure that the elastic sealing member 4 and the lead connection portion 12 are not damaged due to torsional forces in subsequent operations. Subsequently, the second fixing member 32 is further in a locking connection with the first fixing member 31 to continue acting on the elastic sealing member 4, causing the elastic sealing member 4 to be subjected to a compressive force exceeding the second threshold and tightly fit with the first fixing member 31 and the flexible electrode 1, thereby achieving fixation of the flexible electrode 1 and sealing of the wound.

In some embodiments, the flexible electrode 1 includes a first insulating layer, a first conductive layer, and a second insulating layer stacked along the thickness direction of the flexible electrode 1. The electrode site is formed on the first conductive layer, and the first insulating layer and/or the second insulating layer is/are not provided outside a portion of the first conductive layer corresponding to the electrode site.

The first insulating layer and the second insulating layer mentioned above serve to protect the first conductive layer, isolating it from the surrounding target tissue, thereby ensuring accurate transmission of electrical pulses to the electrode site, avoiding the loss of electrical pulses during transmission, and effectively improving the reliability of the flexible neural electrode assembly.

The first insulating layer and second insulating layer mentioned above are made of one or a combination of the following materials: SU-8 photoresist, parylene, fluorinated polymer, and polyimide. In this way, the first and second insulating layers formed of the aforementioned insulating materials can ensure the flexibility and biocompatibility of the flexible neural electrode assembly.

The first conductive layer mentioned above can be made of metal materials with good conductivity and biocompatibility, so that the electrical pulses released by an external apparatus can reach the electrode site. The first conductive layer mentioned above can be made of metal materials such as gold, platinum, iridium, etc., to ensure the effective transmission of electrical signals.

In some embodiments, as shown in FIGS. 1 to 4, a traction portion 21 is formed at the distal end of the traction member 2, traction portion 21 acts on the auxiliary implantation portion 14. The traction member 2 is used to detach the traction portion 21 from the auxiliary implantation portion 14 when subjected to a force applied toward the proximal contact portion 11 of the flexible electrode 1.

In this way, the stabilizing effect on the flexible electrode 1 can be achieved through the traction portion 21 of the traction member 2, ensuring that the flexible electrode 1 can be smoothly implanted into the target tissue.

Embodiments of the present application further provide an implantation method for a flexible neural electrode assembly, which is used for implanting the flexible neural electrode assembly in the above embodiments. The implantation method includes steps S101 to S106.
Step S101: Fix the first fixing member 31 of the auxiliary fixing assembly 3 to the surface of the target tissue and/or the accessory anatomical structure of the target tissue.
Step S102: Implant the distal electrode portion 13 of the flexible electrode 1 and the traction member 2 into the target tissue to a preset depth through the first through channel of the first fixing member 31.
Step S103: Move the elastic sealing member 4 to the proximal end of the first fixing member 31 by applying a force exceeding a first threshold.
Step S104: Separate the traction member 2 from the flexible electrode 1 and remove the traction member 2.
Step S105: Leave the distal electrode portion 13 of the flexible electrode 1 at the preset depth in the target tissue.
Step S106: Connect the second fixing member 32 and the first fixing member 31 via a snap-fit connection or a locking connection, so that the compressive force applied to the elastic sealing member 4 exceeds a second threshold.

Embodiments of the present application further provide an implantation method for a flexible neural electrode assembly, which is used for implanting the flexible neural electrode assembly in the above embodiments. The implantation method includes steps S201 to S206.
Step S201: Fix the first fixing member 31 of the auxiliary fixing assembly 3 to the surface of the target tissue and/or the accessory anatomical structure of the target tissue.
Step S202: Move the elastic sealing member 4 along the length direction of the flexible electrode 1 by applying a force exceeding the first threshold, so that the distal electrode portion 13 of the flexible electrode 1 is located at the preset depth in the target tissue after implantation.
Step S203: Implant the distal electrode portion 13 of the flexible electrode 1 and the traction member 2 into the target tissue to a preset depth through the first through channel of the first fixing member 31, at which point the elastic sealing member 4 reaches the proximal end of the first fixing member 31.
Step S204: Separate the traction member 2 from the flexible electrode 1 and remove the traction member 2.
Step S205: Leave the distal electrode portion 13 of the flexible electrode 1 at the preset depth in the target tissue.
Step S206: Connect the second fixing member 32 and the first fixing member 31 via a snap-fit connection or a locking connection, so that the compressive force applied to the elastic sealing member 4 exceeds the second threshold.

Embodiments of the present application further provide an implantation method for a flexible neural electrode assembly, which is used for implanting the flexible neural electrode assembly in the above embodiments. The implantation method includes steps S301 to S307.
Step S301: Fix the first fixing member 31 of the auxiliary fixing assembly 3 to the surface of the target tissue and/or the accessory anatomical structure of the target tissue.
Step 302: Implant the distal electrode portion 13 of the flexible electrode 1 and the traction member 2 into the target tissue to a preset depth through the first through channel of the first fixing member 31.
Step S303: Move the elastic sealing member 4 to the proximal end of the first fixing member 31 by applying a force exceeding the first threshold.
Step S304: Separate the traction member 2 from the flexible electrode 1 and remove the traction member 2.
Step S305: Leave the distal electrode portion 13 of the flexible electrode 1 at the preset depth in the target tissue.
Step S306: Connect the second fixing member 32 and the first fixing member 31 via the snap-fit connection.
Step S307: Connect the second fixing member 32 and the first fixing member 31 via the locking connection, so that the compressive force applied to the elastic sealing member 4 exceeds the second threshold.

Embodiments of the present application further further provide an implantation method for a flexible neural electrode assembly, which is used for implanting the flexible neural electrode assembly in the above embodiments. The implantation method includes steps S401 to S407.
Step S401: Fix the first fixing member 31 of the auxiliary fixing assembly 3 to the surface of the target tissue and/or the accessory anatomical structure of the target tissue.
Step S402: Move the elastic sealing member 4 along the length direction of the flexible electrode 1 by applying a force exceeding the first threshold, so that the distal electrode portion 13 of the flexible electrode 1 can be located at the preset depth in the target tissue after implantation.
Step S403: Implant the distal electrode portion 13 of the flexible electrode 1 and the traction member 2 into the target tissue to a preset depth through the first through channel of the first fixing member 31, at which point the elastic sealing member 4 reaches the proximal end of the first fixing member 31.
Step S404: Separate the traction member 2 from the flexible electrode 1 and remove the traction member 2.
Step S405: Leave the distal electrode portion 13 of the flexible electrode 1 at the preset depth in the target tissue.
Step S406: Connect the second fixing member 32 and the first fixing member 31 via the snap-fit connection.
Step S407: Connect the second fixing member 32 and the first fixing member 31 via the locking connection, so that the compressive force applied to the elastic sealing member 4 exceeds the second threshold.

Furthermore, although exemplary embodiments have been described herein, their scope comprises any and all embodiments with equivalent elements, modifications, omissions, combinations (e.g., solutions where various embodiments intersect), adaptations, or changes based on the present application. The elements in the claims are to be broadly interpreted based on the language adopted in the claims, and are not limited to the examples described in this specification or during the implementation of the present application, and their examples are to be interpreted as non-exclusive.

The above description is intended to be illustrative rather than limiting. For example, the above examples (or one or more solutions thereof) may be used in combination with each other. For example, other embodiments may be used by those skilled in the art upon reading the above description. In addition, in the above specific embodiments, various features may be grouped together to simplify the present application. This should not be interpreted as an intention that an unclaimed disclosed feature is essential to any claim. On the contrary, the subject matter of the invention may be less than all features of a particular disclosed embodiment. Thus, the claims are incorporated into the detailed description here as examples or embodiments, wherein each claim stands alone as a separate embodiment, and it is considered that these embodiments may be combined with each other in various combinations or permutations. The scope of the invention should be determined with reference to the appended claims along with the full scope of equivalents to which these claims are entitled.

The above embodiments are only exemplary embodiments of the present application, and are not used to limit the invention, and the protection scope of the invention is defined by the claims. Those skilled in the art can make various modifications or equivalent substitutions within the spirit and protection scope of the invention, and such modifications or equivalent substitutions should also be regarded as falling within the protection scope of the invention.

1. A flexible neural electrode assembly comprising:
a flexible electrode made of a flexible material, the flexible electrode comprising a proximal contact portion, a lead connection portion, a distal electrode portion, and an auxiliary implantation portion sequentially connected along a length direction of the flexible electrode, wherein the distal electrode portion and the auxiliary implantation portion are used for being implanted into a target tissue, and the distal electrode portion comprises at least one electrode site for applying electrical stimulation to the target tissue and/or acquiring potential signals of the target tissue;
a traction member acting on the auxiliary implantation portion to implant the flexible electrode toward a target area via the auxiliary implantation portion;
an auxiliary fixing assembly connected to a surface of the target tissue and/or an accessory anatomical structure of the target tissue, wherein the flexible electrode and the traction member both penetrate through the auxiliary fixing assembly for implantation into the target tissue;
an elastic sealing member sleeved outside the flexible electrode and the traction member, wherein the elastic sealing member is configured to abut against the auxiliary fixing assembly at least after the distal electrode portion is implanted into the target tissue to a preset depth, and to deform under the action of the auxiliary fixing assembly to compress the flexible electrode, such that the distal electrode portion of the flexible electrode is fixed at the preset depth, and the flexible electrode is sealingly connected to the auxiliary fixing assembly via the elastic sealing member.

2. The flexible neural electrode assembly according to claim 1, wherein the auxiliary fixing assembly comprises a first fixing member connected to the surface of the target tissue and/or the accessory anatomical structure of the target tissue, and the first fixing member is provided with a first through channel, through which the flexible electrode and the traction member penetrate into the target tissue, and the elastic sealing member abuts against a proximal end of the first fixing member.

3. The flexible neural electrode assembly according to claim 1, wherein the elastic sealing member has a second through channel through which the flexible electrode penetrates, and the second through channel is configured to deform toward the flexible electrode under the action of the auxiliary fixing assembly to abut against the flexible electrode.

4. The flexible neural electrode assembly according to claim 3, wherein the elastic sealing member further has a third through channel through which the traction member penetrates, and the second through channel and the third through channel are in communication with each other or separately arranged.

5. The flexible neural electrode assembly according to claim 1, wherein the elastic sealing member is configured to move relative to the flexible electrode along the length direction of the flexible electrode when subjected to a force exceeding a first threshold.

6. The flexible neural electrode assembly according to claim 4, wherein the second through channel and/or the third through channel is slit-shaped.

7. The flexible neural electrode assembly according to claim 5, wherein the second through channel of the elastic sealing member is configured to apply a first friction force to the flexible electrode penetrating therethrough, and when subjected to a force exceeding the first friction force, the elastic sealing member moves relative to the flexible electrode along the length direction of the flexible electrode;
the third through channel of the elastic sealing member is configured to apply a second friction force to the traction member penetrating therethrough, and when subjected to a force exceeding the second friction force, the elastic sealing member moves relative to the traction member along a length direction of the traction member.

8. The flexible neural electrode assembly according to claim 7, wherein a difference between the first friction force and the second friction force is within a preset range, and the force of the first threshold is configured such that, upon being subjected to the force, the elastic sealing member can overcome the first friction force and the second friction force and move synchronously relative to the flexible electrode and the traction member.

9. The flexible neural electrode assembly according to claim 2, wherein the auxiliary fixing assembly further comprises a second fixing member, which is connected to the first fixing member in a movable manner, and the elastic sealing member is provided between the first fixing member and the second fixing member, and the second fixing member serves to move toward the first fixing member to apply a force to the elastic sealing member to press it against the first fixing member.

10. The flexible neural electrode assembly according to claim 9, wherein the first fixing member and the second fixing member are used to cooperate to apply a compressive force exceeding a second threshold to the elastic sealing member, so that the elastic sealing member can deform to compress the flexible electrode.

11. The flexible neural electrode assembly according to claim 9, wherein the first fixing member and the second fixing member are in a snap-fit connection, and/or the first fixing member and the second fixing member are in a locking connection.

12. The flexible neural electrode assembly according to claim 11, wherein one of the first fixing member and the second fixing member is provided with a recessed portion, and the other is provided with a protruding portion that matches the recessed portion, and the protruding portion is snapped into the recessed portion to achieve the snap-fit connection between the first fixing member and the second fixing member.

13. The flexible neural electrode assembly according to claim 9, wherein the elastic sealing member has a second through channel through which the flexible electrode penetrates, the second fixing member has a fourth through channel through which the flexible electrode penetrates, and the first through channel, the second through channel, and the fourth through channel remain aligned along the length direction of the flexible electrode during connection between the second fixing member and the first fixing member.

14. The flexible neural electrode assembly according to claim 1, wherein the flexible electrode comprises a first insulating layer, a first conductive layer, and a second insulating layer stacked along a thickness direction of the flexible electrode, the electrode site is formed on the first conductive layer, and the first insulating layer and/or the second insulating layer is not provided outside a portion of the first conductive layer corresponding to the electrode site.

15. The flexible neural electrode assembly according to claim 1, wherein a traction portion is formed at a distal end of the traction member, the traction portion acts on the auxiliary implantation portion, and the traction member is used to detach the traction portion from the auxiliary implantation portion when subjected to a force applied toward the proximal contact portion of the flexible electrode.

16. The flexible neural electrode assembly according to claim 2, wherein the elastic sealing member has a second through channel through which the flexible electrode penetrates, and the second through channel is configured to deform toward the flexible electrode under the action of the auxiliary fixing assembly to abut against the flexible electrode.

17. The flexible neural electrode assembly according to claim 11, wherein the elastic sealing member has a second through channel through which the flexible electrode penetrates, the second fixing member has a fourth through channel through which the flexible electrode penetrates, and the first through channel, the second through channel, and the fourth through channel remain aligned along the length direction of the flexible electrode during connection between the second fixing member and the first fixing member.
